(19) United States
(12) Patent Application Publication
(10) Pub. No.: US 2014/0081211 A1
(43) Pub. Date: Mar. 20, 2014

(54) INTRAVENOUS LINE STABILIZATION DEVICE AND METHOD

(71) Applicant: Darrell Lynn Laird, Livingston, TX (US)

(72) Inventor: Darrell Lynn Laird, Livingston, TX (US)

(21) Appl. No.: 14/029,632

(22) Filed: Sep. 17, 2013

Related U.S. Application Data
(60) Provisional application No. 61/743,971, filed on Sep. 17, 2012.
(60) Provisional application No. 61/809,244, filed on Apr. 5, 2013.

(51) Int. Cl. A61M 25/02

(52) U.S. Cl. CPC A61M 25/02
USPC 604/179

(57) ABSTRACT

Described are devices and methods for stabilizing patient IV lines during IV therapy. A sleeve is fitted to a patient and a clasp is placed over IV line(s) at the IV line-receiving area on sleeve, and secured to sleeve via hook-loop or other suitable interactions. In another embodiment, a strap is fitted to the patient. A line strap is attached at one end to strap. IV line(s) is placed in the IV line-receiving area on strap and line strap is secured over IV line(s), attaching at its other end to strap via suitable attachment interactions. In another embodiment, line strap or clasp is removably attachable at both of its ends to strap or sleeve. Anti-slip material can be used on clasp, line strap, and/or receiving area. The strap and line strap may be outfitted with grips to assist in securing, adjusting, and releasing the strap or line strap.

### CROSS-REFERENCE TO RELATED APPLICATIONS

This application claims the benefit of the filing date of and priority to U.S. Provisional Application Ser. No. 61/743,971 entitled “IV wrist stabilizer stable IV wrist and arm Stabilizer” and filed Sep. 17, 2012, Confirmation No. 5328 and to U.S. Provisional Application Ser. No. 61/809,244 entitled “Stable IV Velcro® wrist and arm IV stabilizer” and filed Apr. 5, 2013, Confirmation No. 6028. These provisional applications are incorporated by reference herein.

### STATEMENT REGARDING FEDERALLY SPONSORED RESEARCH OR DEVELOPMENT

Not Applicable.

### BACKGROUND OF THE INVENTION

The present invention is directed apparatuses and methods for stabilizing or securing an intravenous (“IV”) line, central venous catheter line, or any other medical line (collectively, “IV line”) to a desired location on a body of a patient for use during administration of IV liquids in IV therapy.

### BACKGROUND ART

Intravenous therapy is the infusion of a liquid substance directly into the vein of a patient so that the substance can spread through the patient's vascular system. In the field of IV therapy, the use of catheters and IV lines to administer medications, nutrients, volume expanders, blood-based products, and other liquids (collectively, “IV liquids”) to a patient have been well known in the art. With assistance from a health care professional, a needle of a catheter is inserted into a patient's vein, typically located for example, on the wrist, forearm, or any other suitable body location of the patient. A valve that is connected to the catheter is initially set in the closed position to prevent the patient's blood from exiting the catheter upon its insertion into the vein. An IV line, central venous catheter line, or any other medical line (collectively, “IV line”) is connected to the valve of the catheter so that IV liquids can be administered to the patient. The IV line can span several feet in length before it is connected to a source containing IV liquids, typically an IV bag for example.

During IV therapy, it is important to protect both the catheter and the area surrounding the site where the catheter is inserted into the patient's vein (“catheter insertion site”). Because IV lines can span several feet in length to reach the source of IV liquids, they are susceptible to getting pulled, snagged, or tangled, which causes unwanted movement or displacement of the IV line and catheter. In addition, the patient's day-to-day movements can cause the IV line to move or become unstable. An unstable IV line that is free to move can create tensional stress on the catheter as well as the area surrounding the catheter insertion site. If the tensional stress is excessive, the IV line can move, which causes the catheter to move, and can damage the vein by creating a bruise or an infection. Additionally, if tensional stress is excessive, the IV line could potentially become disconnected from the catheter and/or the catheter could potentially be pulled out of the patient unintentionally, causing discontinuance of the IV therapy and potential blood loss from the patient. As such, an unstable IV line has the potential to interfere with the administration of IV liquids.

To prevent the situations described in the preceding paragraph, the IV line must be secured or stabilized to the patient's body to prevent unwanted movement or displacement. Stabilizing the IV line serves many advantages and benefits, including but not limited to: (a) prevents the IV line from getting pulled, snagged, or tangled; (b) prevents the IV line from inadvertently causing tensional stress to the catheter and surrounding area of the catheter insertion site, which may damage the vein; and (c) relieves the patient or health care professional from the burden of having to hold the IV line during administration of IV liquids.

Health care professionals often secure the IV line to the patient's body using adhesive tape or dressing. The adhesive tape or dressing is applied on top of the IV line and directly to the patient's skin. Sometimes, multiple strips of adhesive tape or dressing are needed to stabilize the IV line. However, the use of adhesive tape or dressing is unfavorable for several reasons. First, in the event that the patient's body begins to perspire and create moisture on the surface of the skin, the adhesive in the tape or dressing may fail. Second, adhesive tape or dressing will need to be replaced from time to time due to failure of the adhesive for whatever reason, creating discomfort and inconvenience to the patient. Third, adhesive tape or dressing is not re-usable, creating the need to use new adhesive tape or dressing every time a catheter needs to be inserted, which results in higher consumption. Fourth, adhesive tape or dressing can cause discomfort or annoyance to the patient, because they will pull on the patient's skin and hair upon removal. Fifth, in some cases, the patient's skin may have to be shaved to eliminate obstructive hair before adhesive tape or dressing can be applied in an effort to maximize adhesion to the skin.

Prior art devices exist for securing the catheter to the patient directly over the catheter insertion site. For example, in U.S. Pat. No. 4,898,587 issued to Mera (hereinafter “Mera”), the prior art discloses a “device for securing a catheter on the body adjacent to an incision site in a manner minimizing movement of the catheter with respect to a vein.” (Mera Col. 1 Ln. 8-11.) The device 10 “holds the catheter, the vein into which the catheter is inserted, and the overlying skin of the patient in a fixed relationship to minimize movement of the catheter with respect to the vein.” (Mera Col. 1, Ln 56-58.) Mera further teaches the necessity “to stabilize the catheter to prevent movement which may work the catheter loose and create a potential source of infection or irritation to the patient at the point of catheter insertion.” (Mera Col. 1, Ln. 20-24.) Therefore, the invention disclosed in Mera is used to stabilize the catheter to minimize movement of the catheter, particularly at the catheter insertion site, with the addition of tape, such as described above, to secure the IV line to the patient's arm. However, there remains a need to stabilize the IV line located away from the catheter and catheter insertion site without the use of tape to prevent movement of the catheter at the catheter insertion site. The present invention addresses this need and can actually be used in combination with a device 10 like that shown in Mera. To illustrate, as shown in FIG. 1 of Mera, the present invention can be used in place of tape 72 to secure IV line 20 to patient's arm 14.

In U.S. Pat. No. 4,591,356 titled, “Intravenous Needle Stabilizing Band,” issued to Christie (hereinafter, “Christie”), the prior art discloses “[a]n arm band for stabilizing an intravenous needle inserted into a patient's vein.” (Christie, Abstract)). “By securing the ends of strip 12 together, the encircling edge of hole 14 firmly surrounds intravenous needle 26 at the base thereof adjacent hub 48. Needle 26 is thus effectively and easily secured and immobilized.” (Christie Col. 2, Ln. 26-29.) The arm band “effectively stabilizes and immobilizes an intravenous needle inserted into a patient's vein.” (Christie Col. 2, Ln. 41-43.) Therefore, the invention disclosed in Christie is used to stabilize the needle that is inserted into the patient's vein. In contrast, the present invention is not directed to stabilizing the needle; rather, the present invention is directed to stabilizing the IV line located away from the catheter, which prevents movement of the catheter at the catheter insertion site. The present invention can actually be used in combination with the Christie device. To illustrate, as shown in FIG. 3 of Christie, the present invention can be used to stabilize extended body 46 of tube 24 away from the needle 26 to the patient's arm. Further, as shown in FIG. 3, the Christie device has at least one disadvantage. A hole or fenestra 14 is provided in the body of the strip 12. (Christie Col. 1, Ln. 58-59.) “A slit or cut 18 is made from side edge 16 to fenestra 14, which when unsecured provide an opening for allowing passage therethrough of tube part 24 of intravenous needle 26.” Because of the existence of fenestra 14 and cut 18 to allow needle 26 to pass through strip 12, needle 26 is located underneath strip 12 after it is inserted into the patient's vein. Thus, damage to the vein, including bruising and infection, occurring around needle 26 cannot be seen because of the obstructed view caused by strip 12. However, in using the present invention, there is no slit or cut featured in the device and the device is fitted away from the catheter insertion site. Thus, the catheter insertion site is exposed, and therefore, any damage to the vein is easily visible.

### BRIEF SUMMARY OF THE INVENTION

To address the forgoing problems of the prior art, the present invention is directed to apparatuses and methods for stabilizing or securing IV lines to a desired location on a body of a patient for use during administration of IV liquids in IV therapy.

One embodiment of the invention comprises an expandable, adjustable, and uniformly constructed sleeve is fitted around a desired location on a patient. The sleeve has a sleeve bottom surface for contacting with the desired location on the patient and a sleeve top surface comprising an IV line-receiving area capable of receiving the one or more IV lines. Further, this embodiment has an IV line clasp, comprising an IV line clasp bottom surface, an IV line clasp first end, and an IV line clasp second end. The IV line clasp bottom surface is capable of being placed over the one or more IV lines. Further, the IV line clasp bottom surface at the IV line clasp second end is attachable to the sleeve top surface at the IV line-receiving area. The IV line clasp bottom surface at the IV line clasp first end is attachable to the sleeve top surface, which secures the IV line clasp to the sleeve top surface and over the one or more IV lines.

In another embodiment, the sleeve top surface comprises a loop fastener material and the IV line clasp bottom surface comprises a hook fastener material.

In another embodiment, the sleeve top surface comprises a loop fastener material and the IV line clasp bottom surface comprises a hook fastener material and an anti-slip material, wherein the hook fastener material is located at each end of the IV line clasp, and the anti-slip material is located between the hook fastener material.

In another embodiment, the IV line-receiving area comprises an anti-slip material.

In another embodiment, the sleeve has at least one finger slot.

In another embodiment, the IV line clasp bottom surface at the IV line clasp second end is removably attachable to the sleeve top surface at the IV line-receiving area. Further, the IV line clasp bottom surface at the IV line clasp first end is removably attachable to the sleeve top surface.

In another embodiment, the sleeve top surface comprises a hook fastener material and the IV line clasp bottom surface comprises a loop fastener material and an anti-slip material. The hook fastener material is located at each end of the IV line clasp and the anti-slip material is located between the hook fastener material. The IV line-receiving area comprises an anti-slip material.

In another embodiment, the sleeve top surface comprises a hook fastener material and the IV line clasp bottom surface comprises a loop fastener material. The IV line clasp second end is fixably attached to the sleeve top surface. A grip is attached to the IV line clasp first end. The IV line clasp first end is removably attachable to the sleeve top surface. The IV line-receiving area comprises an anti-slip material.

In another preferred embodiment, the sleeve is a flexible hook-loop strap, comprising a hook-loop strap top surface, a hook-loop strap bottom surface, a hook-loop strap first end, a hook-loop strap second end, and a hook-loop strap interface area, wherein the IV line-receiving area is located on the hook-look strap top surface. The hook-loop strap top surface comprises a hook fastener material and the hook-loop strap bottom surface comprises a loop fastener material. Further, the IV line clasp is a flexible line strap, comprising a line strap bottom surface, a line strap first end, and a line strap second end, wherein the line strap bottom surface comprises a loop fastener material. In addition, the line strap is sewn and attached onto the hook-loop strap with a stitching at the line strap second end, wherein the stitching is located between the IV line-receiving area and the hook-loop strap interface area. The hook-loop strap first end is capable of being wrapped around the desired location of the patient and the hook-loop strap bottom surface adjustably attaches to the hook-loop strap interface area. The line strap is capable of being folded over the IV line-receiving area and over the one or more IV lines. The IV line strap bottom surface is capable of adjustably attaching to the hook-loop strap top surface.

In another embodiment, the line strap bottom surface comprises an anti-slip material and the IV line-receiving area comprises an anti-slip material.

In another embodiment, a first grip is attached to the hook-loop strap first end and a second grip is attached to the line strap first end.

In yet another embodiment of the invention comprises an adjustable and flexible hook-loop strap comprising a hook-loop strap top surface, a hook-loop strap bottom surface, a hook-loop strap first end, a hook-loop strap second end, wherein the hook-loop strap top surface further comprises an IV line-receiving area capable of receiving one or more of the one or more IV lines, and a hook-loop strap interface area. The hook-loop strap top surface comprises a hook fastener material and the hook-loop strap bottom surface comprises a loop fastener material. This embodiment further comprises a flexible line strap attachable to the hook-loop strap, comprising a line strap top surface, a line strap bottom surface, a line strap first end, and a line strap second end, wherein the line strap bottom surface comprises loop fastener material. In addition, this embodiment further comprises a first grip attached to the hook-loop strap first end and a second grip attached to the line strap first end. The line strap bottom surface at the line strap second end is adjustably attachable to the hook-loop strap top surface. The hook-loop strap first end is capable of being wrapped around the desired location of the patient and the hook-loop strap bottom surface adjustably attaches to the hook-loop strap interface area. The line strap is capable of being positioned over the IV line-receiving area and over the one or more IV lines and the line strap bottom surface is capable of being adjustably attached to the hook-loop strap top surface.

In yet another embodiment, the line strap bottom surface at line strap second end and at line strap first end are removably attachable to the hook-loop strap top surface.

In yet another embodiment, the line strap bottom surface at line strap second end is fixably attached to the hook-loop strap top surface.

In yet another embodiment, the line strap bottom surface at line strap second end is fixably attached to the hook-loop strap top surface by sewn stitching or gluing.

In yet another embodiment, the line strap bottom surface comprises an anti-slip material.

In yet another embodiment, the IV line-receiving area comprises an anti-slip material.

In yet another embodiment, the hook-loop strap further comprises a flexible hook strap, comprising a hook strap top surface, a hook strap bottom surface, a hook strap first end, and a hook strap second end, wherein the hook strap top surface comprises the hook fastener material and a flexible loop strap, comprising a loop strap top surface, a loop strap bottom surface, a loop strap first end, and a loop strap second end, wherein the loop strap top surface comprises the loop fastener material. The hook strap bottom surface and the loop strap bottom surface are aligned and adjoined to form an adjustable and flexible hook-loop strap assembly.

In yet another embodiment, the hook-loop strap further comprises a unitary hook-loop strap of uniform construction. The unitary hook-loop strap further comprises a unitary hook-loop strap top surface, a unitary hook-loop strap bottom surface, a unitary hook-loop strap first end, a unitary hook-loop strap second end, wherein the unitary hook strap top surface comprises the hook fastener material and wherein the unitary hook strap bottom surface comprises the loop fastener material.

The preferred embodiments of the apparatuses can be used to practice various methods of stabilizing one or more IV lines to a desired location on a body of a patient undergoing IV therapy. The method assumes that a catheter has been inserted into a vein of the patient at a catheter insertion site and the one or more IV lines has been connected to a valve of the catheter.

One preferred embodiment of the method comprises the steps of placing a flexible and adjustable hook-loop strap on the body of a patient located proximately to but not over the catheter insertion site. The hook-loop strap comprises a hook-loop strap top surface, a hook-loop strap bottom surface, a hook-loop strap first end, a hook-loop strap second end, wherein the hook-loop strap top surface further comprises an IV line-receiving area capable of receiving one or more of the one or more IV lines, and a hook-loop strap interface area. The hook-loop strap top surface comprises a hook fastener material and the hook-loop strap bottom surface comprises a loop fastener material. A flexible line strap is attachable to the hook-loop strap, comprising a line strap top surface, a line strap bottom surface, a line strap first end, and a line strap second end, wherein the line strap bottom surface comprises loop fastener material. A first grip is attached to the hook-loop strap first end and a second grip is attached to the line strap first end. The line strap bottom surface at the line strap second end is attachable to the hook-loop strap top surface. Also, the hook-loop strap first end is capable of being wrapped around the desired location of the patient and the hook-loop strap bottom surface adjustably attaches to the hook-loop strap interface area. The line strap is capable of being positioned over the IV line-receiving area and over the one or more IV lines and the line strap bottom surface is capable of being adjustably attached to the hook-loop strap top surface.

Next, the hook-loop strap is wrapped around the desired location of the patient. Next, the hook-loop strap bottom surface at the hook-loop strap first end is adjustably attached to the hook-loop strap interface area so that the hook-loop strap is fitted to the patient. After that, one or more IV lines are placed in the IV line-receiving area. The line strap is then placed over the one or more IV lines and over the IV line-receiving area. Finally, the line strap bottom surface is adjustably secured to the hook-loop strap top surface.

The use of the present invention solves many of the problems associated with the prior art's use of adhesive tape and dressing. For example, perspiration and moisture caused by the patient's body would not affect the use of the invention. In addition, the present invention is durable and does not need to be replaced as frequently as tape or dressing as discussed in the prior art. Further, the present invention is not a disposable product and can be re-used numerous times for the same patient or other patients. Moreover, the present invention will not cause the patient to experience discomfort or annoyance upon its removal since it does not use any adhesive contacting the patient's body, eliminating the chance of pulling on the patient's skin and hair upon its removal. Also, since the present invention does not require use of any adhesive, the patient's hair located where the present invention is to be fitted to the body, does not need to be shaved.

### BRIEF DESCRIPTION OF THE DRAWINGS

FIG. 1A is a perspective view of one embodiment of the present invention of the loop strap only, showing loop strap top surface comprising loop fastener material.

FIG. 1B is a perspective view of one embodiment of the present invention of the hook strap only, showing hook strap top surface comprising hook fastener material. The hook strap is preferably the same or approximately the same length as the loop strap shown in FIG. 1A.

FIG. 1C is a perspective view of one embodiment of the present invention of the line strap only.

FIG. 1D is a perspective view of one embodiment of the present invention of the primary grip only, showing the primary grip cut-out hole.

FIG. 1E is a perspective view of one embodiment of the present invention of the secondary grip only, showing the secondary grip cut-out hole.

FIG. 2A is a top view of the loop strap, showing that loop strap top surface comprises loop fastener material.

FIG. 2B is a profile view of the loop strap, showing that loop strap top surface comprises loop fastener material.

FIG. 3A is a top view of the hook strap, showing that hook strap top surface comprises hook fastener material. The hook strap is preferably the same or approximately the same length as the line strap shown in FIG. 2A.

FIG. 3B is a profile view of the hook strap, showing that hook strap top comprises hook fastener material.

FIG. 4A is a top view of the line strap.

FIG. 4B is a profile view of the line strap, showing that line strap bottom surface comprises loop fastener material.

FIG. 5A is a top view of the primary grip.

FIG. 5B is a profile view of the primary grip, showing the primary grip cut-out hole.

FIG. 6A is a top view of the secondary grip.

FIG. 6B is a profile view of the secondary grip, showing the secondary grip cut-out hole.

FIG. 7 is a perspective view of one embodiment of the present invention wherein the loop strap and the hook strap are adjoined at each of their respective bottom surfaces to form a hook strap, also referred to as a hook-loop strap assembly. The primary grip is attached to the hook-loop strap assembly. The secondary grip is attached to the line strap. The line strap is attached to the hook-loop strap assembly with a stitching. In this drawing, the hook-loop strap assembly is in a closed position and the line strap is in a closed position.

FIG. 8 depicts the insertion of a catheter into a patient's vein at the catheter insertion site. An IV line runs from the valve of the catheter. The hook-loop strap assembly is adjustably wrapped around the forearm of the patient in a closed position, away from the catheter insertion site using one embodiment of the present invention. The line strap is shown in a closed position where it stabilizes the IV line to the forearm of the patient.

FIG. 9 shows a perspective front view of one embodiment of the present invention, wherein the hook-loop strap assembly is in an open position and the line strap is in a closed position. The IV-line receiving area, hook-loop interface area, primary grip, secondary grip, and stitching are also shown.

FIG. 10 shows a perspective rear view of one embodiment of the present invention, wherein the hook-loop strap assembly is in an open position and the line strap is in a closed position.

FIG. 11 is a close-up view of one embodiment of the present invention of the hook-loop strap assembly in an open position and the line strap in an open position showing the IV line-receiving area and also showing the line strap bottom surface made entirely of loop fastener material. The hook-loop interface area is also shown.

FIG. 12 is a close-up view of one embodiment of the present invention of the hook-loop strap assembly in an open position and the line strap in an open position showing the IV line-receiving area and also showing the line strap bottom surface made partially of loop fastener material and anti-slip material. The hook-loop interface area is also shown.

FIG. 13 is a close-up view of one embodiment of the present invention of the hook-loop strap assembly in an open position and the line strap in an open position showing the IV line-receiving area with anti-slip material and also showing the line strap bottom surface made entirely of loop fastener material. The hook-loop interface area is also shown.

FIG. 14 is a close-up view of one embodiment of the present invention of the hook-loop strap assembly in an open position and the line strap in an open position showing the IV line-receiving area with anti-slip material and also showing the line strap bottom surface made partially of loop fastener material and anti-slip material. The hook-loop interface area is also shown.

FIG. 15 is a close-up view of one embodiment of the present invention showing the line strap bottom surface made partially of loop fastener material and anti-slip material, wherein the anti-slip material is located between the loop fastener material.

FIG. 16 is a close-up view of one embodiment of the present invention showing the line strap bottom surface made partially of loop fastener material and anti-slip material, wherein the anti-slip material is located from the line strap second end until it becomes adjacent to the loop fastener material.

FIG. 17 shows a perspective front view of one embodiment of the present invention, wherein the hook-loop strap, also referred to as a unitary hook-loop strap, is of uniform construction. The unitary hook-loop strap is in an open position and the line strap is in a closed position. The IV-line receiving area, hook-loop interface area, primary grip, secondary grip, and stitching are also shown.

FIG. 18 shows a perspective rear view of one embodiment of the present invention, wherein the unitary hook-loop strap is in an open position and the line strap is in a closed position.

FIG. 19 is a close-up view of one embodiment of the present invention of the unitary hook-loop strap in an open position and the line strap in an open position showing the IV line-receiving area and also showing the line strap bottom surface made entirely of loop fastener material. The hook-loop interface area is also shown.

FIG. 20 is a close-up view of one embodiment of the present invention of the unitary hook-loop strap in an open position and the line strap in an open position showing the IV line-receiving area and also showing the line strap bottom surface made partially of loop fastener material and anti-slip material. The hook-loop interface area is also shown.

FIG. 21 is a close-up view of one embodiment of the present invention of the unitary hook-loop strap in an open position and the line strap in an open position showing the IV line-receiving area with anti-slip material and also showing the line strap bottom surface made entirely of loop fastener material. The hook-loop interface area is also shown.

FIG. 22 is a close-up view of one embodiment of the present invention of the unitary hook-loop strap in an open position and the line strap in an open position showing the IV line-receiving area with anti-slip material and also showing the line strap bottom surface made partially of loop fastener material and anti-slip material. The hook-loop interface area is also shown.

FIG. 23A is a perspective view of one embodiment of the present invention showing a solid primary grip, which does not contain a primary grip cut-out hole.

FIG. 23B is a perspective view of one embodiment of the present invention showing a solid secondary grip, which does not contain a secondary grip cut-out hole.

FIG. 24 is a front view of one embodiment of the present invention where a uniformly constructed sleeve and an IV line clasp are provided. The sleeve is fitted over the palm of a patient, wherein the sleeve has at least one finger slot for the patient's finger, preferably the patient's thumb, to extend through. The IV line clasp is placed over an IV line and attaches to the sleeve at one orientation and is in a closed position.

FIG. 25 is a front view of one embodiment of the present invention where a uniformly constructed sleeve and an IV line clasp are provided. The sleeve is fitted over the palm of a patient, wherein the sleeve has at least one finger slot for the patient's finger, preferably the patient's thumb, to extend through. The IV line clasp is placed over an IV line and attaches to the sleeve at a different orientation relative to the orientation shown in FIG. 24 and is in a closed position.

FIG. 26 is a perspective view of one embodiment of the present invention of the IV line clasp only, where the IV line clasp bottom surface is made entirely of hook fastener material.

FIG. 27 is a perspective view of one embodiment of the present invention of the IV line clasp only, where the IV line clasp bottom surface is made partially of hook fastener material and anti-slip material, wherein the anti-slip material is located between the hook fastener material.

FIG. 28 is a perspective view of one embodiment of the present invention where a uniformly constructed sleeve and an IV line clasp are provided. The IV line clasp bottom surface is placed over the IV line and attaches to the sleeve top surface and is in a closed position.

FIG. 29 is a perspective view of one embodiment of the present invention where a uniformly constructed sleeve and an IV line clasp are provided. The IV line clasp is attached to sleeve top surface with stitching at IV line clasp second end. A primary grip is attached to IV line clasp first end. The IV line clasp bottom surface is placed over the IV line and attaches to the sleeve top surface. The IV line clasp is in a closed position.

### DETAILED DESCRIPTION OF THE INVENTION

Reference is now made to the drawings, which depict preferred embodiments of the present invention, but are not drawn to scale.

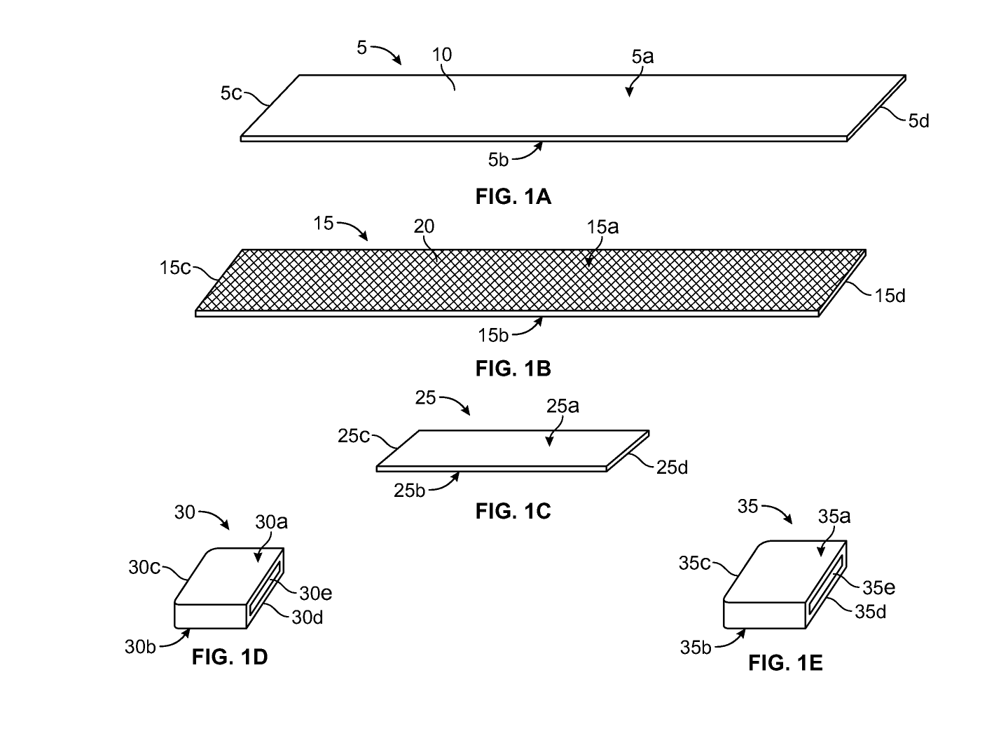

As shown in FIGS. 1A-1E, the main components of one preferred embodiment of the present invention comprise the following: loop strap 5, hook strap 15, line strap 25, primary grip 30, and secondary grip 35.

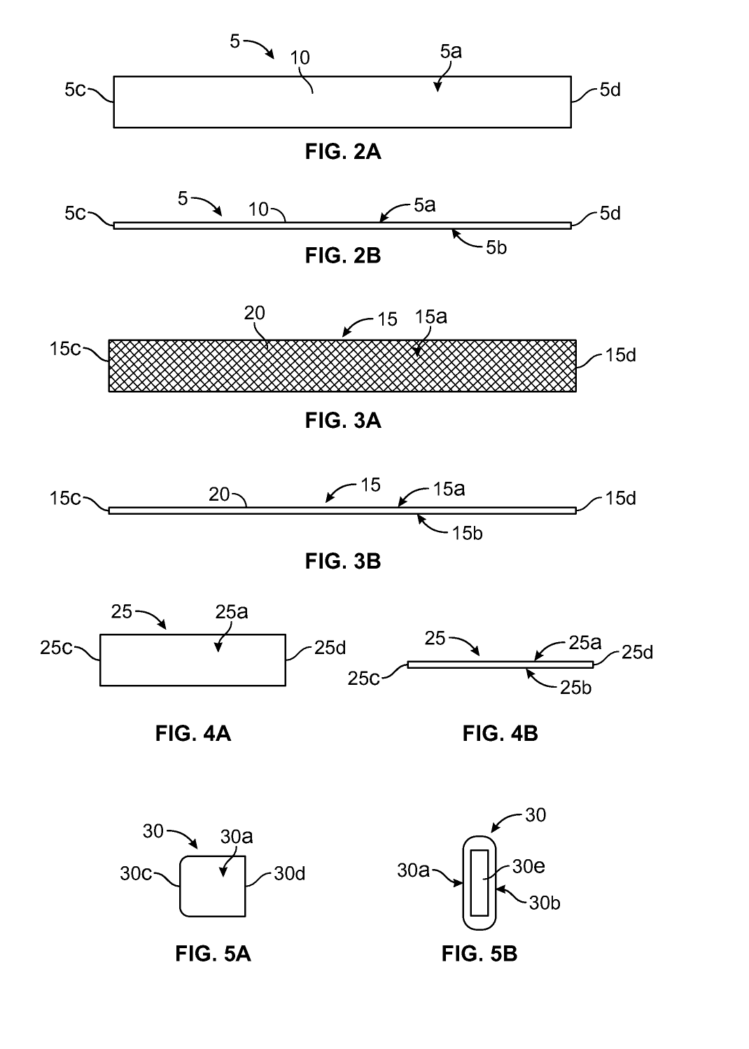

Referring to FIGS. 1A, 2A, and 2B, loop strap 5 comprises loop strap top surface 5a, loop strap bottom surface 5b, loop strap first end 5c, and loop strap second end 5d. Loop strap top surface 5a comprises loop fastener material 10.

Referring to FIGS. 1B, 3A, and 3B, hook strap 15 comprises hook strap top surface 15a, hook strap bottom surface 15b, hook strap first end 15c, and hook strap second end 15d. Hook strap top surface 15a comprises hook fastener material 20.

Referring to FIGS. 1C, 4A, and 4B, line strap 25 comprises line strap top surface 25a, line strap bottom surface 25b, line strap first end 25c, and line strap second end 25d. Line strap bottom surface 25b comprises loop fastener material 10.

Referring to FIGS. 1D, 5A, and 5B, primary grip 30 comprises primary grip top surface 30a, primary grip bottom surface 30b, primary grip first end 30c, primary grip second end 30d, and primary grip cut-out hole 30e. Primary grip cut-out hole 30e is located proximate primary grip second end 30d. Primary grip cut-out hole 30e is an area of empty space located within the interior volume of primary grip 30. Primary grip 30 may be made of any suitable material, comprising for example, rubber, plastic, or any other polymer known in the art, or metal, and is preferably constructed to provide nonslip characteristics on its surface to aid in gripping. In a preferred embodiment, primary grip 30 is constructed of plastic. The actual primary grip 30 design could also have contoured surfaces (not shown) or have surface features, e.g. ridges, divots, dimples (not shown) that aid in gripping.

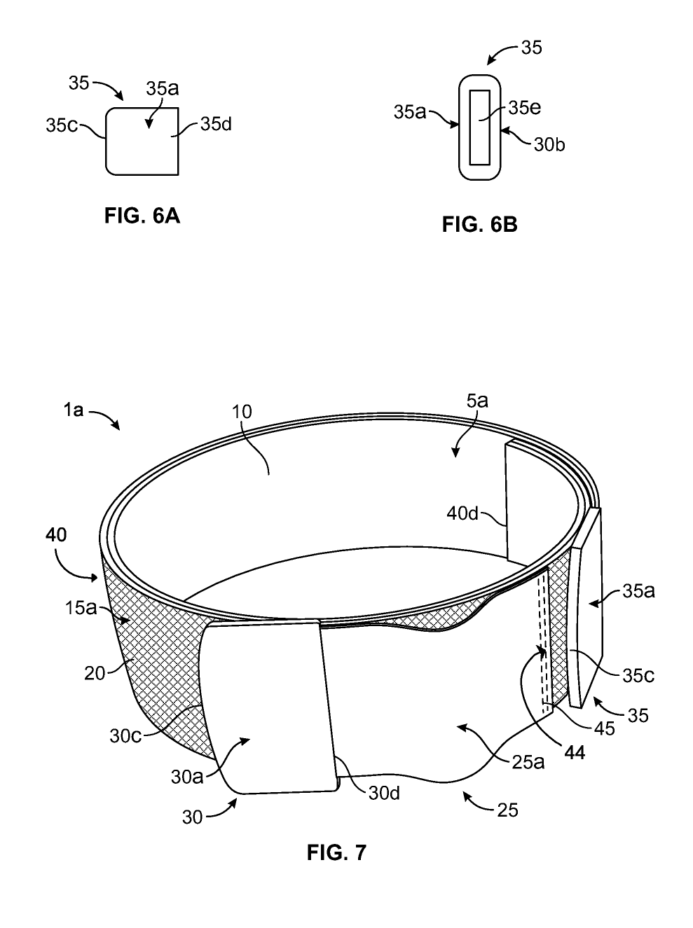

Referring to FIGS. 1E, 6A, and 6B, secondary grip 35 comprise secondary grip top surface 35a, secondary grip bottom surface 35b, secondary grip first end 35c, secondary grip second end 35d, and secondary grip cut-out hole 35e. Secondary grip cut-out hole 35e is located proximate secondary grip second end 35d. Secondary grip cut-out hole 35e is an area of empty space located within the interior volume of secondary grip 35. Secondary grip 35 may be made of any suitable material, comprising for example, rubber, plastic, or any other polymer known in the art, or metal, and is preferably constructed to provide nonslip characteristics on its surface to aid in gripping. In a preferred embodiment, secondary grip 35 is constructed of plastic. The actual secondary grip 35 design could also have contoured surfaces (not shown) or have surface features, e.g. ridges, divots, dimples (not shown) that aid in gripping. Preferably, secondary grip 35 comprises the same nonslip material as primary grip 30.

As seen in FIG. 7, loop strap 5, hook strap 15, line strap 25, primary grip 30, and secondary grip 35 are assembled together to form one preferred embodiment of the present IV line stabilization device 1a invention, as further shown in FIGS. 9-11 and discussed below.

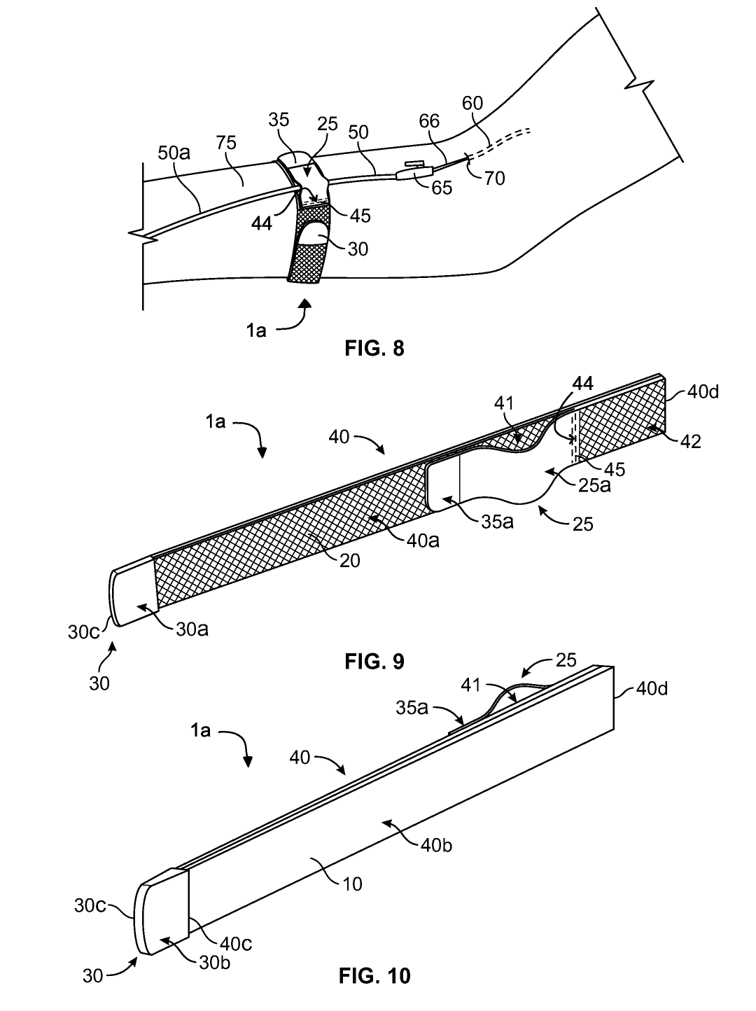

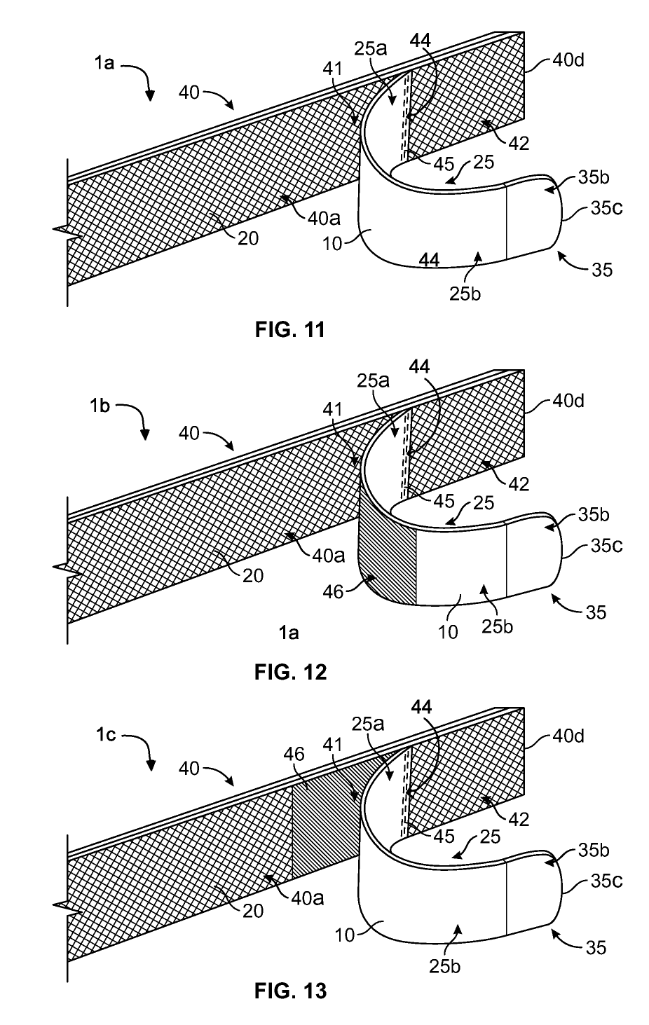

Referring to FIG. 8, forearm 75 of patient is prepared for administration of IV liquids. Catheter 60, which is connected to valve 65 of catheter 60, is inserted into vein 61 at catheter insertion site 70 using any method known in the art. IV line 50, which is desired to be stabilized, is attached to valve 65 of catheter 60 using any method known in the art. As discussed below, in one preferred embodiment, the hook-loop strap, also referred to as hook-loop strap assembly 40, is adjustably wrapped to forearm 75 away from catheter insertion site 70. IV line 50 is placed on top of IV line-receiving area 41 (see FIGS. 11-14). By folding line strap 25 over IV line-receiving area 41 (see FIGS. 11-14) and over IV line 50, IV line 50 is stabilized or secured to forearm 75 of patient. As further shown in FIG. 8, in the event that IV line liquid source end 50a experiences tensional stress from pulling, tugging, or snagging, then catheter 60 and catheter insertion site 70 remain protected or undisturbed and will not cause damage to vein 61, allowing administration of IV liquids without interference or disruption because IV line 50 is stabilized.

In one preferred embodiment, and referring to FIGS. 1-8, loop strap 5 and hook strap 15 are preferably the same or approximately the same length. Loop strap bottom surface 5b (see FIGS. 1A, 2B) is adjoined to hook strap bottom surface 15b (see FIGS. 1B, 3B) to form hook-loop strap, also referred to as hook-loop strap assembly 40, using any means known in the art, such as the use of glue or other adhesive. As shown in FIGS. 9-10, hook-loop strap or hook-loop strap assembly 40 comprises hook-loop strap assembly top surface 40a, hook-loop strap assembly bottom surface 40b, hook-loop strap assembly first end 40c, hook-loop strap assembly second end 40d, IV line-receiving area 41, and hook-loop strap interface area 42. Hook-loop strap assembly top surface 40a comprises hook fastener material 20. Hook-loop strap assembly bottom surface 40b comprises loop fastener material 10. Primary grip 30 is inserted and attached to hook-loop strap assembly 40 using any means known in the art such as the use of glue or other adhesive, wherein primary grip cut-out hole 30e (shown in FIGS. 1D and 5B) is inserted into hook-loop strap assembly first end 40c and attached. Secondary grip 35 is inserted and attached to line strap 25 using any means known in the art such as the use of glue or other adhesive, wherein secondary grip cut-out hole 35e (shown in FIGS. 1E and 6B) is inserted into line strap first end 25c (shown in FIGS. 4A and 4B) and attached.

Referring back to FIGS. 4A and 4B, line strap 25 is fixably or removably attached at line strap second end 25d to hook-loop strap assembly 40 at attachment area 44 to create a hinge-like attachment using any suitable attachment techniques known in the art, such as for example, sewing, gluing, riveting, snaps, hook and loop fastener material (such as those offered under the Velcro® brand), and the like. Attachment area 44 is located between IV line-receiving area 41 and hook-loop strap interface area 42, as shown in FIGS. 9-14.

As shown in FIGS. 7-14, in various preferred embodiments, line strap 25 is fixably attached to hook-loop strap assembly 40, by sewing stitching 45 proximate to line strap second end 25d and sewing stitching 45 through hook-loop strap assembly 40 at attachment area 44. Preferably stitching 45 is a double stitch, but may be any other type of stitching known in the art to provide a secure attachment of line strap 25 to hook-loop strap assembly 40.

In another embodiment (not shown), line strap 25 is removably attached to hook-loop strap assembly 40. For example, line strap bottom surface 25b comprises loop fastener material 10 proximate line strap first end 25c and line strap second end 25d. Line strap second end 25d is removably attached to hook fastener material 40 of hook-loop strap assembly top surface 40a via hook-loop interactions” or the like. As used herein, hook-loop interactions or the like means the interactive forces that bind hook fastener material 20 and loop fastener material 10 together (and vice-versa), when the materials come into contact with each other, as well known in the art. In such an embodiment, line strap 25 can be attached to hook-loop strap assembly top surface 40a, removed, and then re-attached to hook-loop strap assembly top surface 40a. This embodiment provides the freedom of attaching line strap 25 to different locations to hook-loop strap top surface 40a as desired.

Referring back to FIG. 8, hook-loop strap or hook-loop strap 40 is wrapped and adjustably fitted around forearm 75 and away from catheter insertion site 70 and not covering catheter insertion site 70. As shown in FIGS. 9-10, loop fastener material 10 of hook-loop strap assembly bottom surface 40b attaches to hook fastener material 20 of hook-loop strap assembly top surface 40a via hook-loop interactions occurring at hook-loop strap interface area 42. The present invention is said to be adjustable and can be adjustably fitted to a desired location on the body of the patient, because hook-loop strap assembly bottom surface 40b can be selectively attached to various locations of hook-loop interface area 42 to accommodate various sizes or girths of patient's body parts and limbs (e.g. hand, palm, forearm, wrist, arm, thigh, leg, calf) and accommodate for any swelling experienced by the patient's body parts or limbs. The use of hook fastener material 20 and loop fastener material 10 make it easy for the present invention to be quickly adjusted and fitted to the desired location of the patient. Optionally, grasping primary grip 30 and pulling with sufficient force, breaks the hook-loop interactions to allow for the quick removal or adjustment of hook-loop strap assembly 40 on the desired location on the patient.

Referring back to the embodiments shown in FIGS. 7-10, after placing IV line 50, which is desired to be stabilized, over IV line-receiving area 41, line strap 25 is folded about stitching 45, whereby loop fastener material 10 of line strap bottom surface 25b is attached to hook fastener material 20 of hook-loop strap assembly top surface 40a via hook-loop interactions. As a result, IV line 50 is stabilized or secured to forearm 75. Optionally, grasping secondary grip 35 and pulling with sufficient force, breaks the hook-loop interaction to allow for the quick removal of IV line 50 or re-securement of IV line 50 to a stabilized position.

FIG. 11 represents IV line stabilization device 1a as shown in FIGS. 7-10. FIG. 11 shows hook-loop strap assembly 40 in an open position and line strap 25 in an open position to further show line strap bottom surface 25b, which is made entirely of loop fastener material 10. With line strap 25 in an open position, IV line-receiving area 41 is exposed and located on hook-loop strap assembly top surface 40a and below line strap 25. In this embodiment, IV line-receiving area 41 is made entirely of hook fastener material 20. Secondary grip 35 is shown attached to line strap first end 25c (shown in FIGS. 4A and 4B).

Referring to FIG. 12, IV line stabilization device 1b is shown in another preferred embodiment of the invention. FIG. 12 shows hook-loop strap assembly 40 in an open position and line strap 25 in an open position to further show line strap bottom surface 25b, which comprises loop fastener material 10 and anti-slip material 46. Anti-slip material 46 comprises any material that provides a nonslip surface or the like, and is preferable made of rubber or any other polymer that comprises a nonslip surface as known in the art. Anti-slip material 46 is used to eliminate or reduce the slipping or sliding of IV line 50 while it is being stabilized. Referring back to FIG. 12, in this preferred embodiment, IV line-receiving area 41 is made entirely of hook fastener material 20. Secondary grip 35 is shown attached to line strap first end 25c (shown in FIGS. 4A and 4B).

Referring to FIG. 13, IV line stabilization device 1c is shown in another preferred embodiment of the invention. FIG. 13 shows hook-loop strap assembly 40 in an open position and line strap 25 in an open position to further show line strap bottom surface 25b, which is made entirely of loop fastener material 10. With line strap 25 in an open position, IV line-receiving area 41 is exposed and located on hook-loop strap assembly top surface 40a below line strap 25. In this preferred embodiment, IV line-receiving area 41 comprises anti-slip material 46. Secondary grip 35 is shown attached to line strap first end 25c (shown in FIGS. 4A and 4B).

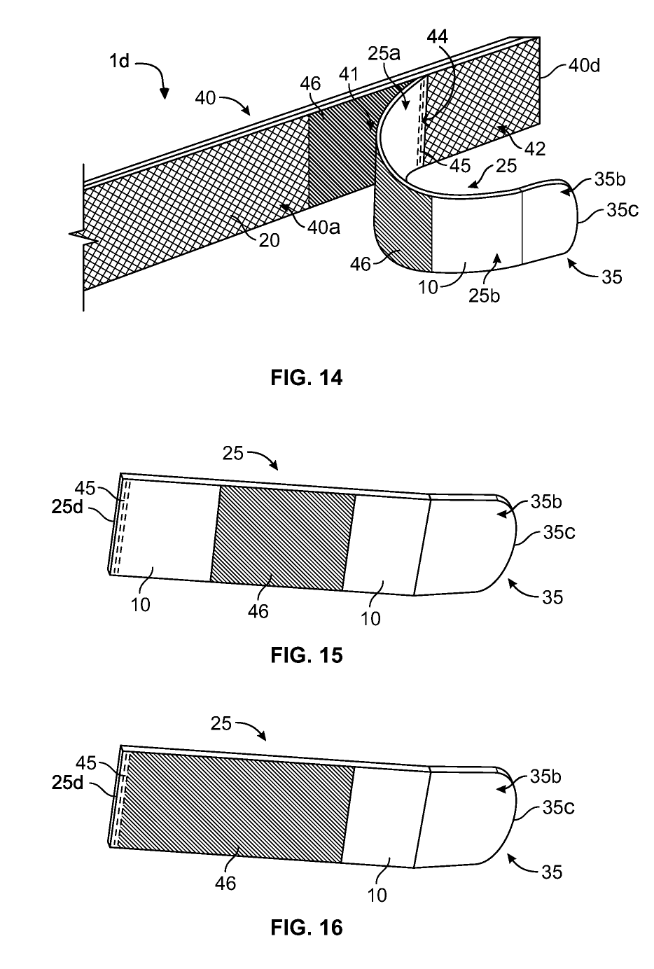

Referring to FIG. 14, IV line stabilization device 1d is shown in another preferred embodiment of the invention. FIG. 14 shows hook-loop strap assembly 40 in an open position and line strap 25 in an open position to further show line strap bottom surface 25b, which is made partially of loop fastener material 10 and anti-slip material 46. With line strap 25 in an open position, IV line-receiving area 41 is exposed and located on hook-loop strap assembly top surface 40a below line strap 25. In this preferred embodiment, IV line-receiving area 41 comprises anti-slip material 46. Secondary grip 35 is shown attached to line strap first end 25c (shown in FIGS. 4A and 4B).

Referring to FIGS. 15 and 16, anti-slip material 46 can be found on line strap bottom surface 25b at various locations in different embodiments of the present invention. Referring to FIG. 15 and back to FIGS. 4A and 4B, line strap bottom surface 25b is shown of line strap 25. Line strap bottom surface 25b is shown partially of loop fastener material 10 and anti-slip material 46. In this embodiment and referring to FIGS. 15, 4A, and 4B, anti-slip material 46 is located on line strap bottom surface 25b at the center of line strap bottom surface 25b, whereby loop fastener material 10 is located adjacent to anti-slip material 46 at line strap first end 25c and line strap second end 25d as shown. Secondary grip 35 is shown attached to line strap first end 25c. Stitching 45 is shown attached to line strap second end 25d.

Referring to FIG. 16 and back to FIGS. 4A and 4B, line strap bottom surface 25b is shown of line strap 25. Line strap bottom surface 25b is shown partially of loop fastener material 10 and anti-slip material 46. In this embodiment and referring FIGS. 16, 4A, and 4B, anti-slip material 46 is located on line strap bottom surface 25b beginning from line strap second end 25d and through and until ant-slip material 46 contacts with loop fastener material 10 as shown. Secondary grip 35 is shown attached to line strap first end 25c. Stitching 45 is shown attached to line strap second end 25d.

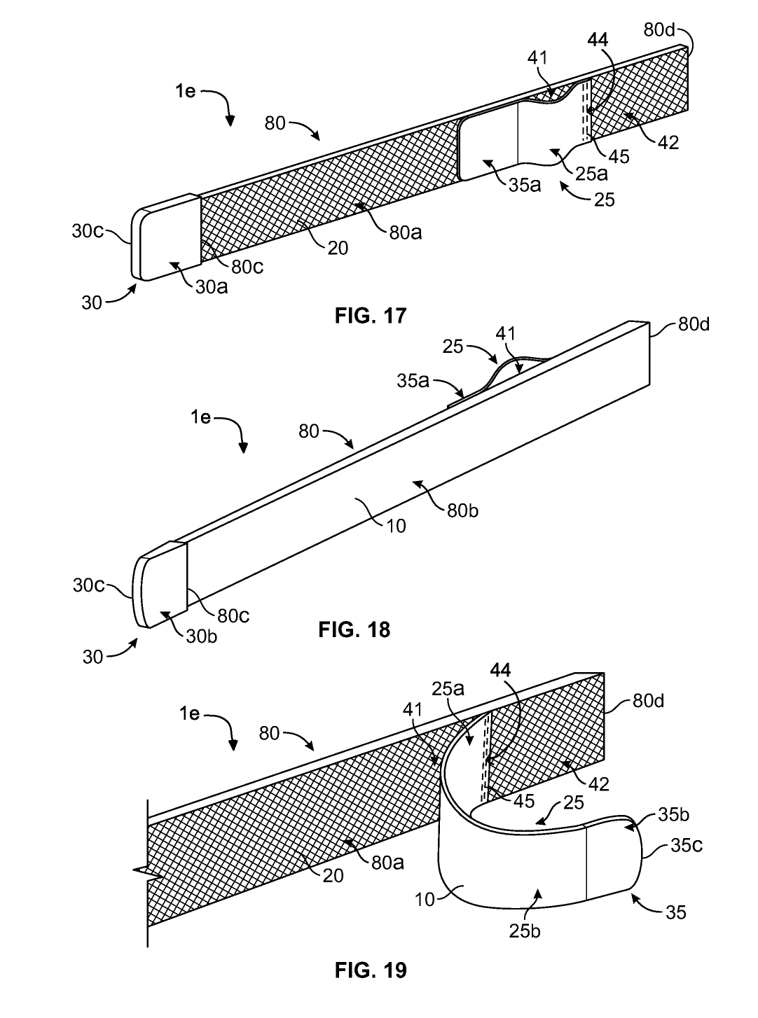

Additional embodiments of the present invention are disclosed, wherein the hook strap is of a uniform construction, using a single strap that comprises hook fastener material 20 on one surface and loop fastener material 10 on the opposite surface. FIGS. 17-18, for example, show another embodiment of IV line stabilization device 1e, which uses a hook-strap of uniform construction. Hook-loop strap, also referred to as unitary hook-loop strap 80, can be used in lieu of hook-loop strap assembly 40 as shown in FIGS. 9-14. In this embodiment, hook loop strap or unitary hook-loop strap 80 has unitary hook-loop strap top surface 80a, unitary hook-loop strap bottom surface 80b, unitary hook-loop strap first end 80c, and unitary hook-loop strap second end 80d. Unitary hook-loop strap top surface 80a comprises hook fastener material 20. Unitary hook-loop strap bottom surface 80b comprises loop fastener material 10. Primary grip 30 is inserted and attached to unitary hook-loop strap 80 using any means known in the art such as the use of glue or other adhesive, wherein primary grip cut-out hole 30e (shown in FIGS. 1D and 5B) is inserted into unitary hook-loop strap first end 80c and attached. Secondary grip 35 is attached to line strap 25 using any means known in the art such as the use of glue or other adhesive, wherein secondary grip cut-out hole 35e (shown in FIGS. 1E and 6B) is inserted into line strap first end 25c (shown in FIGS. 4A and 4B) and attached.

Referring back to FIGS. 4A and 4B, line strap 25 is fixably or removably attached at line strap second end 25d to unitary hook-loop strap 80 at attachment area 44 to create a hinge-like attachment using any suitable attachment techniques known in the art, such as for example, sewing, gluing, riveting, snaps, hook and loop fasteners, and the like. Attachment area 44 is located between IV line-receiving area 41 and hook-loop strap interface area 42, as shown in FIGS. 17-22.

As shown in FIGS. 17-22, in one preferred embodiment, line strap 25 is fixably attached, by sewing stitching 45 proximate to line strap second end 25d and sewing stitching 45 through unitary hook-loop strap 80 at attachment area 44. Preferably stitching 45 is a double stitch, but may be any other type of stitching known in the art to provide a secure attachment of line strap 25 to unitary hook-loop strap 80.

In another embodiment (not shown), line strap 25 is removably attached unitary hook-loop strap 80. For example, line strap bottom surface 25b comprises loop fastener material 10 proximate line strap first end 25c and line strap second end 25d. Line strap second end 25d is removably attached to hook fastener material 40 of unitary hook-loop strap top surface 80a via hook-loop interactions. In this embodiment, line strap 25 can be attached to unitary hook-loop strap top surface 80a, removed, and then re-attached to unitary hook-loop strap top surface 80a. This embodiment provides the freedom of attaching line strap 25 to different locations to unitary hook-loop strap top surface 80a as desired.

Unitary hook-loop strap 80 is wrapped around and adjustably fitted to forearm 75 and away from catheter insertion site 70 and not covering catheter insertion site 70. As shown in FIGS. 17-18, loop fastener material 10 of unitary hook-loop strap bottom surface 80b attaches to hook fastener material 20 of unitary hook-loop strap top surface 80a via hook-loop interactions occurring t hook-loop strap interface area 42. The present invention is said to be adjustable and can be adjustably fitted to a desired location on the body of the patient, because unitary hook-loop strap bottom surface 80b can be selectively attached to various locations of hook-loop interface area 42 to accommodate various sizes of patient's forearm 75 or other body or limb of patient. The use of hook fastener material 20 and loop fastener material 10 make it easy for the present invention to be quickly adjusted and fitted to the desired location on the body of the patient. Optionally, grasping primary grip 30 and pulling with sufficient force, breaks the hook-loop interactions to allow for the quick removal or adjustment of unitary hook-loop strap 80 on the desired location on the body of the patient.

After placing IV line 50, which is desired to be stabilized, over IV line-receiving area 41, line strap 25 is folded about stitching 45, whereby loop fastener material 10 of line strap bottom surface 25b is attached to hook fastener material 20 of unitary hook-loop strap top surface 80a via hook-loop interactions. As a result, IV line 50 is stabilized or secured to forearm 75. Optionally, grasping secondary grip 35 and pulling with sufficient force, breaks the hook-loop interaction to allow for the quick removal of IV line 50 or re-securement of IV line 50 to a stabilized position.

FIG. 19 represents IV line stabilization device 1e as shown in FIGS. 17-18. FIG. 19 shows unitary hook-loop strap 80 in an open position and line strap 25 in an open position to further show line strap bottom surface 25b, which is made entirely of loop fastener material 10. With line strap 25 in an open position, IV line-receiving area 41 is exposed and located on unitary hook-loop strap top surface 80a and below line strap 25. In this embodiment, IV line-receiving area 41 is made entirely of hook fastener material 20. Secondary grip 35 is shown attached to line strap first end 25c (shown in FIGS. 4A and 4B).

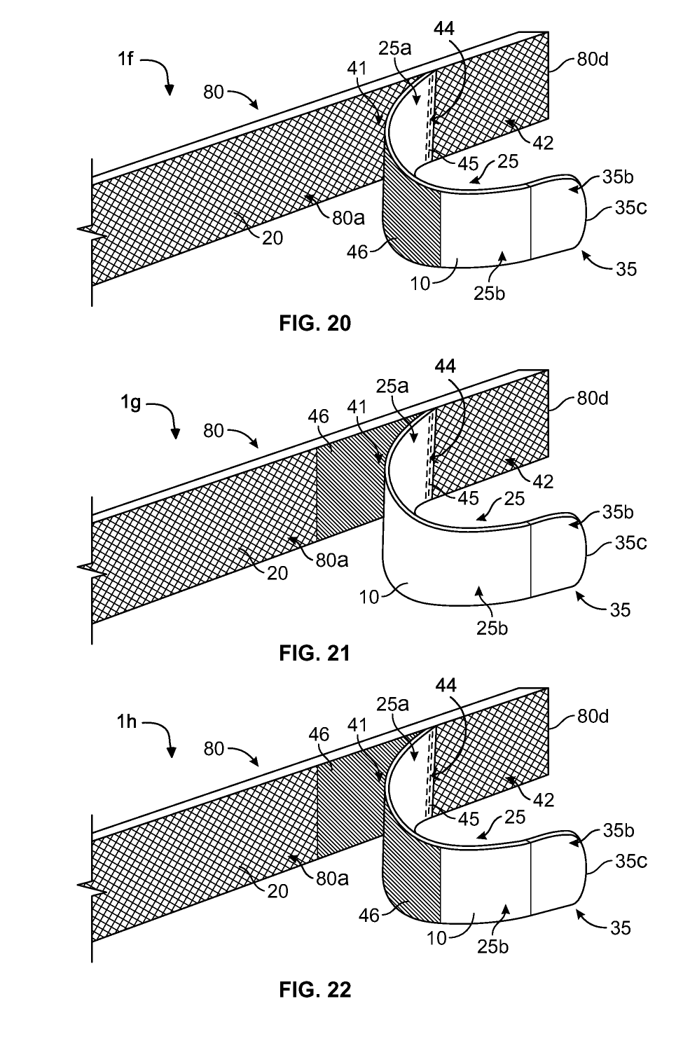

Referring to FIG. 20, IV line stabilization device 1f is shown in another preferred embodiment of the invention. FIG. 20 shows unitary hook-loop strap 80 in an open position and line strap 25 in an open position to further show line strap bottom surface 25b, which comprises loop fastener material 10 and anti-slip material 46. Anti-slip material 46 comprises any material that provides a nonslip surface or the like, and is preferable made of rubber or any other polymer that comprises a nonslip surface as known in the art. Anti-slip material 46 is used to eliminate or reduce the slipping or sliding of IV line 50 while it is being stabilized. Referring back to FIG. 20, in this preferred embodiment, IV line-receiving area 41 is made entirely of hook fastener material 20. Secondary grip 35 is shown attached to line strap first end 25c (shown in FIGS. 4A and 4B).

Referring to FIG. 21, IV line stabilization device 1g is shown in another embodiment of the invention. FIG. 21 shows unitary hook-loop strap 80 in an open position and line strap 25 in an open position to further show line strap bottom surface 25b, which is made entirely of loop fastener material 10. With line strap 25 in an open position, IV line-receiving area 41 is exposed and located on unitary hook-loop strap top surface 80a below line strap 25. In this preferred embodiment, IV line-receiving area 41 comprises anti-slip material 46. Secondary grip 35 is shown attached to line strap first end 25c (shown in FIGS. 4A and 4B).

Referring to FIG. 22, IV line stabilization device 1h is shown in another preferred embodiment of the invention. FIG. 22 shows unitary hook-loop strap 80 in an open position and line strap 25 in an open position to further show line strap bottom surface 25b, which is made partially of loop fastener material 10 and anti-slip material 46. With line strap 25 in an open position, IV line-receiving area 41 is exposed and located on unitary hook-loop strap top surface 80a below line strap 25. In this preferred embodiment, IV line-receiving area 41 comprises anti-slip material 46. Secondary grip 35 is shown attached to line strap first end 25c (shown in FIGS. 4A and 4B).

As discussed above, in certain embodiments of the present IV line stabilization device (i.e. 1f and 1h) invention that use hook strap or unitary hook-loop strap 80, line strap 25 can have anti-slip material 46 on line strap bottom surface 25b, wherein anti-slip material 46 can be found on line strap bottom surface 25b at various locations as previously shown in FIGS. 15-16 and previously discussed.

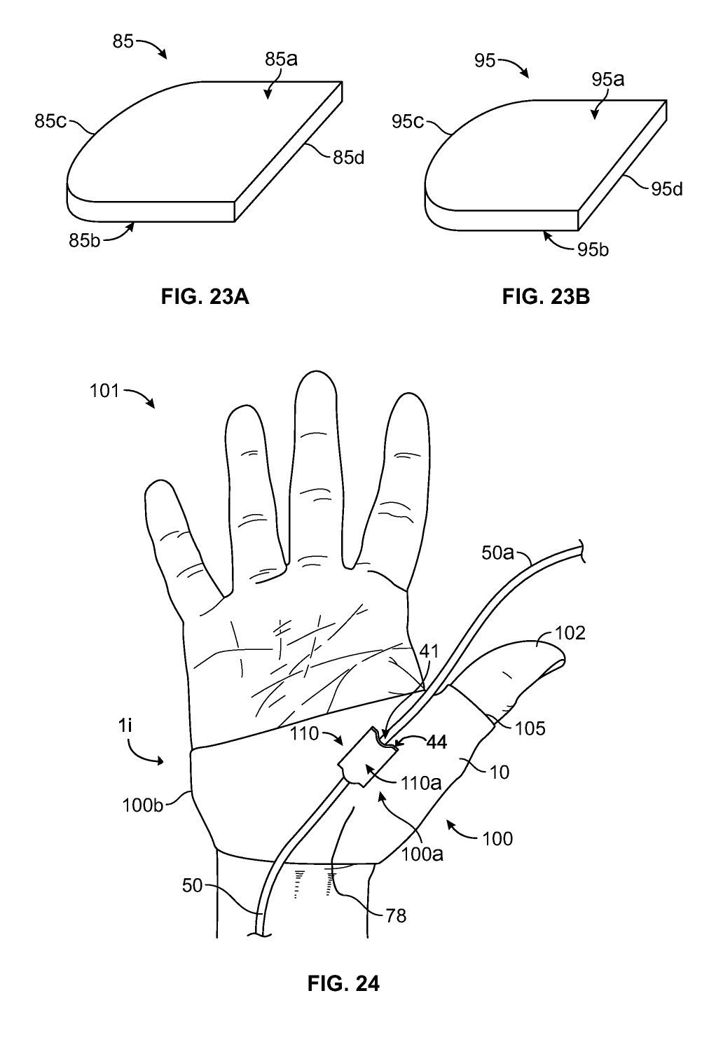

Referring to FIG. 23A, solid primary grip 85 is shown. Solid primary grip 85 is essentially primary grip 30 without primary grip cut-out hole 30e as shown in FIGS. 1D and 5B. Referring to FIG. 23A, solid primary grip 85 comprises solid primary grip top surface 85a, solid primary grip bottom surface 85b, solid primary grip first end 85c, and solid primary grip second end 85d. In additional preferred embodiments (not shown), solid primary grip 85 can be attached to hook-loop strap assembly first end 40, unitary hook-loop strap first end 80c, line strap first end 25c, or line strap second end 25d, by use of glue or other adhesive known in the art at solid primary grip bottom surface 85b proximate solid primary grip second end 85d.

Referring to FIG. 23B, solid secondary grip 95 is shown. Solid secondary grip 95 is essentially secondary grip 35 without secondary grip cut-out hole 35e as shown in FIGS. 1E and 6B. Referring to FIG. 23B, solid secondary grip 95 comprises solid secondary grip top surface 95a, solid secondary grip bottom surface 95b, solid secondary grip first end 95c, and solid secondary grip second end 95d. In additional preferred embodiments (not shown), solid secondary grip 95 can be attached to hook-loop strap assembly first end 40, unitary hook-loop strap first end 80c, line strap first end 25c, or line strap second end 25d, by use of glue or other adhesive known in the art at solid secondary grip bottom surface 95b proximate solid secondary grip second end 85d.

Referring to FIG. 24, another embodiment of the IV line stabilization device 1i invention is shown. An expandable, adjustable, and uniformly constructed sleeve 100 comprises, sleeve top surface 100a, sleeve bottom surface 100b, finger slot 105, and IV line-receiving area 41. Sleeve 100 is uniformly constructed because it has no slit or cut through sleeve bottom surface 100b or sleeve top surface 100a. Sleeve 100 comprises elastic material as known in the art, thereby making it expandable and adjustable so that it can be fitted to a desired location on the patient to accommodate for various sizes or girths of a patient's body parts and limbs (e.g. hand, palm, forearm, wrist, arm, thigh, leg, calf) and accommodate for any swelling experienced by the patient's body parts or limbs. In one embodiment, sleeve 100 is fitted through patient's hand 101 and over patient's palm 78, whereby sleeve bottom surface 100b contacts palm 78, and whereby sleeve 100 is fitted away from catheter insertion site 70 (not shown) and not over catheter insertion site 70 (not shown). Sleeve top surface 100a comprises loop fastener material 10 and is capable of receiving one or more IV lines 50 at IV line-receiving area 41. Finger 102, preferably the patient's thumb, is extended through finger slot 105. IV line 50, which is desired to be stabilized, is placed on top of palm 78 over IV line-receiving area 41. As shown in FIGS. 26-27, IV line clasp 110 comprises IV line clasp top surface 110a, IV line clasp bottom surface 110b, IV line clasp first end 110c, and IV line clasp second end 110d.

In one embodiment of the present invention (not shown), IV line clasp bottom surface 110b comprises entirely of hook fastener material 20. Referring back to FIG. 24, IV line clasp 110 is placed over IV line 50 and IV line clasp bottom surface 110b (shown in FIG. 27) and removably attaches to sleeve top surface 100a proximate IV line clasp first end 110c and IV line clasp second end 110d, using hook-loop interactions, to stabilize IV line 50 in one orientation to palm 78. As illustrated in FIG. 24, IV line clasp 110 is shown in the closed position. In the event that IV line liquid source end 50a experiences tensional stress from pulling, tugging, or snagging, then catheter 60 (not shown) and catheter insertion site 70 (not shown) remain protected or undisturbed and will not cause damage to vein 61 (not shown), allowing administration of IV liquids without interference or disruption because IV line 50 is stabilized. IV line clasp 50 can be removed and re-attached to sleeve top surface 100a.

In another preferred embodiment (not shown), primary grip 30 (see FIGS. 1D, 5A, and 5B) could be attached to IV line clasp first end 110c. IV line clasp first end 110c can be inserted and attached to primary grip cut-out hole 30e using any methods known in the art such as using glue or adhesive. Primary grip 30 can be used to assist in securing or releasing IV line 50 about IV line clasp first end 110c.

In another preferred embodiment (not shown), secondary grip 35 (see FIGS. 1E, 6A, and 6B) could be attached to IV line clasp second end 110d. IV line clasp second end 110d can be inserted and attached to secondary grip cut-out hole 35e using any techniques known in the art such as glue or adhesive. Secondary grip 35 can be used to assist in securing or releasing IV line 50 about IV line clasp second end 110d.

In yet another preferred embodiment (not shown), primary grip 30 can be attached to IV line clasp first end 110c and secondary grip 35 can be attached to IV line clasp second end 110d. Primary grip 30 and secondary grip 35 can be used to assist in securing or releasing IV line 50 about IV line clasp first end 110c and IV line clasp second end 110d, respectively.

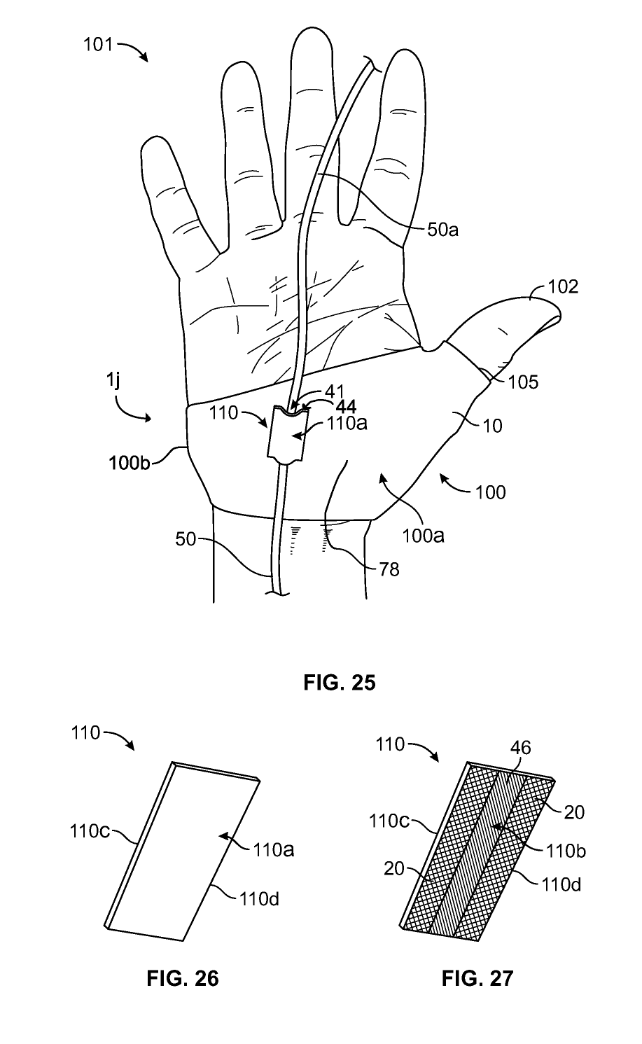

Referring to FIG. 25, IV line 50 can be secured or stabilized to palm 78 at different orientations and locations for desired reasons, including for example, to promote comfort for patient. IV line clasp bottom surface 110b of IV line clasp 110 can be removably attached to sleeve top surface 100a at various orientation and locations through hook-loop interactions. As illustrated in FIG. 25, IV line clasp 110 is shown in the closed position, but oriented in a different position as IV line clasp 110 shown in FIG. 24.

Referring to FIGS. 26-27, IV line clasp 110 has IV line clasp top surface 110a, IV line clasp bottom surface 110b, IV line clasp first end 110c, and IV line clasp second end 110d. IV line clasp 110 is used to stabilize or secure IV line 50. In one embodiment of the invention, as shown in FIG. 27, IV line clasp bottom surface 110b is made partially of hook fastener material 20 and anti-slip material 46. Anti-slip material 46 comprises any material that provides a nonslip surface or the like, and is preferable made of rubber or any other polymer that comprises a nonslip surface as known in the art. Anti-slip material 46 is used to eliminate or reduce the slipping or sliding of IV line 50 while it is being stabilized. Referring to FIG. 27, anti-slip material 46 is contained in the center of IV line clasp bottom surface 110b, and located at IV line clasp first end 110c and IV line clasp second end 110d, and situated between hook fastener material 20. Hook fastener material 20 of IV line clasp bottom surface 110b attaches to sleeve top surface 100a via hook-loop interactions. As a result, IV line 50 is secured or stabilized to palm 78 as shown in FIGS. 24-25 and previously discussed.

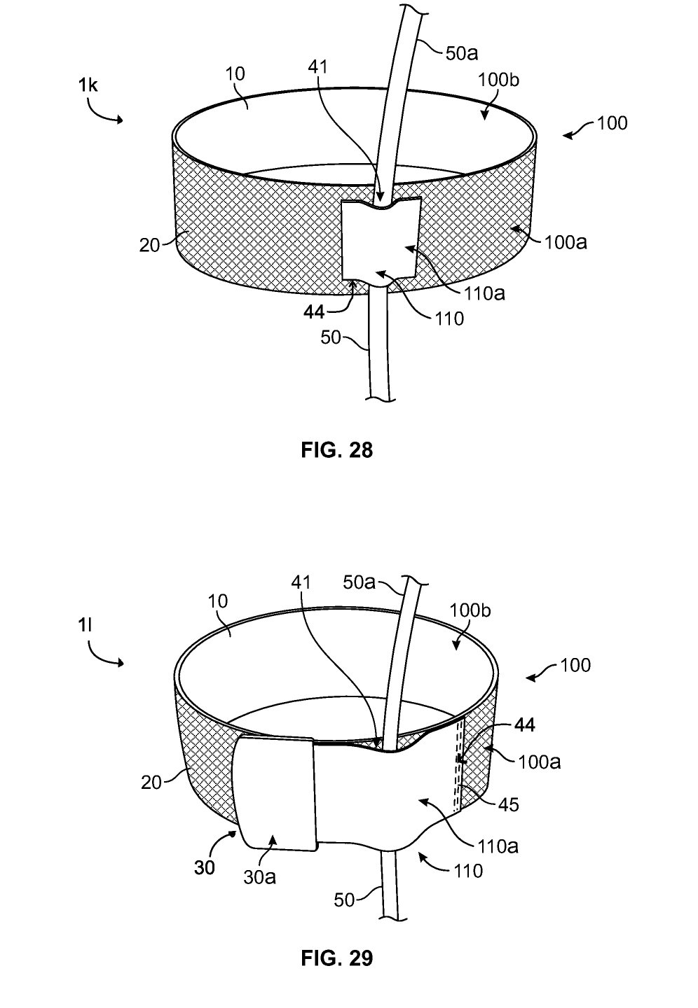

Referring back to FIGS. 26-27, IV line clasp 110 comprises IV line clasp top surface 110a, IV line clasp bottom surface 110b, IV line clasp first end 110c, and IV line clasp second end 110d. In the preferred embodiment shown in FIG. 28, IV line clasp bottom surface 110b (not shown) comprises loop fastener material 10. IV line 50 is placed over IV line-receiving area 41. IV line clasp bottom surface 110b (not shown) of IV line clasp 110 is placed over IV line 50 and over IV line-receiving area 41 and adjustably attaches to sleeve top surface 100a. Through hook-loop interactions, IV line 50 is secured or stabilized to the desired location on the body of the patient. As illustrated in FIG. 28, IV line clasp 110 is shown in the closed position. In the event that IV line liquid source end 50a experiences tensional stress from pulling, tugging, or snagging, then catheter 60 (not shown) and catheter insertion site 70 (not shown) remain protected or undisturbed and will not cause damage to vein 61 (not shown), allowing administration of IV liquids without interference or disruption because IV line 50 is stabilized.

In another preferred embodiment (not shown), primary grip 30 (see FIGS. 1D, 5A, and 5B) could be attached to IV line clasp first end 110c. IV line clasp first end 110c can be inserted and attached to primary grip cut-out hole 30e using any methods known in the art such as using glue or adhesive. Primary grip 30 can be used to assist in securing or releasing IV line 50 about IV line clasp first end 110c.

In another preferred embodiment (not shown), secondary grip 35 (see FIGS. 1E, 6A, and 6B) could be attached to IV line clasp second end 110d. IV line clasp second end 110d can be inserted and attached to secondary grip cut-out hole 35e using any techniques known in the art such as glue or adhesive. Secondary grip 35 can be used to assist in securing or releasing IV line 50 about IV line clasp second end 110d.

In yet another preferred embodiment (not shown), primary grip 30 can be attached to IV line clasp first end 110c and secondary grip 35 can be attached to IV line clasp second end 110d. Primary grip 30 and secondary grip 35 can be used to assist in securing or releasing IV line 50 about IV line clasp first end 110c and IV line clasp second end 110d, respectively.

Referring back to FIGS. 26-27, IV line clasp 110 has IV line clasp top surface 110a, IV line clasp bottom surface 110b, IV line clasp first end 110c, and IV line clasp second end 110d. In the preferred embodiment shown in FIG. 29, IV line clasp bottom surface 110b (not shown) comprises loop fastener material 10. IV line clasp 110 is fixably attached to sleeve top surface 100a, by sewing stitching 45 proximate to IV line clasp second end 110d and sewing stitching 45 through sleeve 100 at attachment area 44. Preferably stitching 45 is a double stitch, but may be any other type of stitching known in the art to provide a secure attachment of IV line clasp 110 to sleeve top surface 100a. IV line 50 is placed over IV line-receiving area 41. IV line clasp 110 is placed over IV line 50 and over IV line-receiving area 41. IV line clasp 110 is folded about stitching 45 over IV line 50 and over IV line-receiving area 41, where IV line clasp bottom surface 110b attaches to sleeve top surface 100a through hook-loop interactions. Through hook-loop interactions, IV line 50 is secured or stabilized to the desired location on the body of the patient. IV line clasp 110 is shown in the closed position. In the event that IV line liquid source end 50a experiences tensional stress from pulling, tugging, or snagging, then catheter 60 (not shown) and catheter insertion site 70 (not shown) remain protected or undisturbed and will not cause damage to vein 61 (not shown), allowing administration of IV liquids without interference or disruption because IV line 50 is stabilized.

Referring to FIG. 28, IV line stabilization device 1k is shown in another preferred embodiment of the invention. An expandable, adjustable, and uniformly constructed sleeve 100 comprises, sleeve top surface 100a, sleeve bottom surface 100b, and IV line-receiving area 41. Sleeve 100 is uniformly constructed because it has no slit or cut through sleeve bottom surface 100b or sleeve top surface 100a. Sleeve 100 comprises elastic material as known in the art, thereby making it expandable and adjustable so that it can be fitted to a desired location on the patient to accommodate for various sizes or girths of a patient's body parts and limbs (e.g. hand, palm, forearm, wrist, arm, thigh, leg, calf) and accommodate for any swelling experienced by the patient's body parts or limbs. Sleeve 100 is fitted through a desired location on the patient away from catheter insertion site 70 (not shown) and not over catheter insertion site 70 (not shown). Sleeve top surface 100a comprises hook fastener material 20 uniformly distributed throughout sleeve top surface 100a as shown. Optionally, sleeve bottom surface 100b comprises loop fastener material 10 or any other material suitable for contacting the body of the patient.

In another preferred embodiment (not shown), primary grip 30 (see FIGS. 1D, 5A, and 5B) could be attached to IV line clasp first end 110c. IV line clasp first end 110c can be inserted and attached to primary grip cut-out hole 30e using any methods known in the art such as using glue or adhesive. Primary grip 30 can be used to assist in securing or releasing IV line 50 about IV line clasp first end 110c.

In another preferred embodiment (not shown), secondary grip 35 (see FIGS. 1E, 6A, and 6B) could be attached to IV line clasp second end 110d. IV line clasp second end 110d can be inserted and attached to secondary grip cut-out hole 35e using any techniques known in the art such as glue or adhesive. Secondary grip 35 can be used to assist in securing or releasing IV line 50 about IV line clasp second end 110d.

In yet another preferred embodiment (not shown), primary grip 30 can be attached to IV line clasp first end 110c and secondary grip 35 can be attached to IV line clasp second end 110d. Primary grip 30 and secondary grip 35 can be used to assist in securing or releasing IV line 50 about IV line clasp first end 110c and IV line clasp second end 110d, respectively.

Referring to FIG. 29, IV line stabilization device 11 is shown in yet another preferred embodiment of the invention. An expandable, adjustable, and uniformly constructed sleeve 100 comprises, sleeve top surface 100a, sleeve bottom surface 100b, and IV line-receiving area 41. Sleeve 100 is uniformly constructed because it has no slit or cut through sleeve bottom surface 100b or sleeve top surface 100a. Sleeve 100 comprises elastic material as known in the art, thereby making it expandable and adjustable so that it can be fitted to a desired location on the patient to accommodate for various sizes or girths of a patient's body parts and limbs (e.g. hand, palm, forearm, wrist, arm, thigh, leg, calf) and accommodate for any swelling experienced by the patient's body parts or limbs. In one embodiment, sleeve 100 is fitted through a desired location on the patient and away from catheter insertion site 70 (not shown) and not over catheter insertion site 70 (not shown). Sleeve top surface 100a comprises hook fastener material 20 uniformly distributed throughout sleeve top surface 100a as shown. Optionally, sleeve bottom surface 100b comprises loop fastener material 10 or any other material suitable for contacting the body of the patient.

In other embodiments (not shown), sleeve top surface 100a described and shown in FIGS. 24-25 and 28-29 may comprise hook fastener material 20 selectively distributed on sleeve top surface 100a as one or more hook fastener material segments positioned on such surfaces (e.g., in a spaced apart relationship) to facilitate positioning of the fastening and facilitate the elasticity and stretching of the embodiments shown in FIGS. 24-25 and 28-29.

In another preferred embodiment (shown in FIG. 29), primary grip 30 (see FIGS. 1D, 5A, and 5B) could be attached to IV line clasp first end 110c. IV line clasp first end 110c can be inserted and attached to primary grip cut-out hole 30e using any methods known in the art such as using glue or adhesive. Primary grip 30 can be used to assist in securing or releasing IV line 50 about IV line clasp first end 110c.

In another preferred embodiment (not shown), secondary grip 35 (see FIGS. 1E, 6A, and 6B) could be attached to IV line clasp first end 110c. IV line clasp first end 110c can be inserted and attached to secondary grip cut-out hole 35e using any techniques known in the art such as glue or adhesive. Secondary grip 35 can be used to assist in securing or releasing IV line 50 about IV line clasp first end 110c.

If desired, additional IV line clasps 110 could be secured to sleeve top surfaces 100a of various embodiments (e.g., those shown in FIGS. 24, 25, 28 and 29) to provide additional orientations for securing one or more IV lines 50.

It will be apparent to one of ordinary skill in the art having the benefit of the present disclosure that additional embodiments of the invention can be derived. For example, the embodiments illustrated in FIGS. 9-14 and 17-22 could be further modified wherein line strap 25 is removably attachable at line strap first end 25c and line strap second end 25d, similar to how IV line clasp 110 is removably attachable at IV line clasp 110c and IV line clasp 110d as shown FIGS. 24, 25, and 28. Optionally, line strap first end 25c could be attached to primary grip 30 and line strap second end 110d could be attached to secondary grip 35 similar to how IV line clasp first end 110c could be attached to primary grip 30 and IV line clasp second end 110d could be attached to secondary grip 35.

In addition, it will be apparent to one of ordinary skill in the art having the benefit of the present disclosure that additional embodiments of the invention can be derived, whereby loop fastener material 10 can be used in place of hook fastener material 20 and whereby hook fastener material 20 can be used in place of loop fastener material 20.

In addition, it will be apparent to one of ordinary skill in the art having the benefit of the present disclosure that additional embodiments of the invention can be derived, any of the grips described can be of other grip-like structures known in the art.

The present invention is also directed to methods of stabilizing one or more IV lines 50 to a desired location on a body of a patient undergoing IV therapy. The methods assume that a catheter 66 has been inserted into a vein 60 of the patient at a catheter insertion site 70 and one or more IV lines 50 has been connected to a valve 65 of catheter 66 as shown in FIG. 8. Using one embodiment of the present IV line stabilization device, IV line stabilization device 1a for example as shown in FIGS. 8-9, the method comprises the steps of placing flexible and adjustable hook loop strap, also referred to as hook-loop strap assembly 40, on a body of a patient located proximately to but not over catheter insertion site 70. Next, hook-loop strap or hook-loop strap assembly 40 is adjustably attached to hook-loop strap bottom surface 40b proximate to the hook-loop strap first end 40c to the hook-loop strap interface area 42 so that hook-loop strap assembly 40 is fitted to the patient. Next, one or more IV lines 50 is placed in IV line-receiving area 41. After that, line strap 25 is placed over the one or more IV lines 50 and over IV line-receiving area 41. Finally, line strap bottom surface 25b is adjustably secured to hook-loop strap top surface 40a. The result of the method is that in the event that IV line liquid source end 50a experiences tensional stress from pulling, tugging, or snagging, then catheter 60 and catheter insertion site 70 remain protected or undisturbed and will not cause damage to vein 61, allowing administration of IV liquids without interference or disruption because IV line 50 is stabilized. It will be apparent to one of ordinary skill in the art having the benefit of the present disclosure that additional methods using different embodiments of the invention can be disclosed. For example, the method can be used in conjunction with IV line stabilization devices 1b through 1h.

In addition, other methods of stabilizing one or more IV lines 50 to a desired location on a patient undergoing IV therapy can be practiced in conjunction with other embodiments of IV line stabilization device, IV line stabilization devices 1i through 1l, for example. The method assumes that a catheter 66 has been inserted into a vein 60 of the patient at a catheter insertion site and one or more IV lines 50 has been connected to a valve 65 of catheter 66. The method comprises the steps of placing an expandable, adjustable, and uniformly constructed sleeve 100 to fit around a desired location of a patient located proximately to but not over catheter insertion site 70. Next, one or more IV lines 50 is placed over IV line-receiving area 41. After that, IV line clasp 110 is placed over the one or more IV lines 50 and over IV line-receiving area 41. Finally, IV line clasp bottom surface 110b is adjustably secured to sleeve top surface 100a. The result of the method is that in the event that IV line liquid source end 50a experiences tensional stress from pulling, tugging, or snagging, then catheter 60 and catheter insertion site 70 remain protected or undisturbed and will not cause damage to vein 61, allowing administration of IV liquids without interference or disruption because IV line 50 is stabilized.

### REFERENCES

The following represents an exemplary list of references.

### U.S. Patent References

1. U.S. Pat. No. 4,898,587 issued to Mera; and
2. U.S. Pat. No. 4,591,356 issued to Christie.

All references referred to herein are incorporated herein by reference. While the apparatuses and methods of this invention have been described in terms of preferred embodiments, it will be apparent to those of skill in the art that variations may be applied to the apparatuses and methods described herein without departing from the concept and scope of the invention. All such similar substitutes and modifications apparent to those skilled in the art are deemed to be within the scope and concept of the invention. Those skilled in the art will recognize that the apparatuses and methods of the present invention have many applications, and that the present invention is not limited to the representative examples disclosed herein. Moreover, the scope of the present invention covers conventionally known variations and modifications to the components described herein, as would be known by those skilled in the art. While the apparatuses and methods of this invention have been described in terms of preferred or illustrative embodiments, it will be apparent to those of skill in the art that variations may be applied to the process described herein without departing from the concept and scope of the invention. All such similar substitutes and modifications apparent to those skilled in the art are deemed to be within the scope and concept of the invention as it is set out in the following claims.

I claim:

1. An IV line stabilization device used to stabilize one or more IV lines to a desired location on a body of a patient undergoing IV therapy, comprising:
(a) an expandable, adjustable, and uniformly constructed sleeve to fit around the desired location of the patient;
the sleeve having a sleeve bottom surface for contacting with the desired location of the patient and a sleeve top surface comprising an IV line-receiving area capable of receiving the one or more IV lines; and
(b) an IV line clasp comprising an IV line clasp bottom surface, an IV line clasp first end, and an IV line clasp second end, wherein the IV line clasp bottom surface is capable of being placed over the one or more IV lines, wherein the IV line clasp bottom surface at the IV line clasp second end is attachable to the sleeve top surface at the IV line-receiving area, and wherein the IV line clasp bottom surface at the IV line clasp first end is attachable to the sleeve top surface, to secure the IV line clasp to the sleeve top surface and over the one or more IV lines.

2. The IV line stabilization device of claim 1, wherein the sleeve top surface comprises a loop fastener material and the IV line clasp bottom surface comprises a hook fastener material.

3. The IV line stabilization device of claim 1, wherein the sleeve top surface comprises a loop fastener material and the IV line clasp bottom surface comprises a hook fastener material and an anti-slip material, wherein the hook fastener material is located at each end of the IV line clasp, and the anti-slip material is located between the hook fastener material.

4. The IV line stabilization device of claim 1 wherein the IV line-receiving area comprises an anti-slip material.

5. The IV line stabilization device of claim 1 wherein the sleeve has at least one finger slot.

6. The IV line stabilization device of claim 1 wherein the IV line clasp bottom surface at the IV line clasp second end is removably attachable to the sleeve top surface at the IV line-receiving area, and wherein the IV line clasp bottom surface at the IV line clasp first end is removably attachable to the sleeve top surface.

7. The IV line stabilization device of claim 1, wherein the sleeve top surface comprises a hook fastener material and the IV line clasp bottom surface comprises a loop fastener material and an anti-slip material, wherein the hook fastener material is located at each end of the IV line clasp, and the anti-slip material is located between the hook fastener material, and wherein the IV line-receiving area comprises an anti-slip material.

8. The IV line stabilization device of claim 1, wherein the sleeve top surface comprises a hook fastener material and the IV line clasp bottom surface comprises a loop fastener material, wherein the IV line clasp second end is fixably attached to the sleeve top surface, wherein a grip is attached to the IV line clasp first end, wherein the IV line clasp first end is removably attachable to the sleeve top surface, and wherein the IV line-receiving area comprises an anti-slip material.

9. The IV line stabilization device of claim 1,
(a) wherein the sleeve is a flexible hook-loop strap, comprising a hook-loop strap top surface, a hook-loop strap bottom surface, a hook-loop strap first end, a hook-loop strap second end, and a hook-loop strap interface area, wherein the IV line-receiving area is located on the hook-look strap top surface;
(b) wherein the hook-loop strap top surface comprises a hook fastener material and the hook-loop strap bottom surface comprises a loop fastener material;
(c) wherein the IV line clasp is a flexible line strap, comprising a line strap bottom surface, a line strap first end, and a line strap second end, wherein the line strap bottom surface comprises a loop fastener material;
(d) wherein the line strap is sewn and attached onto the hook-loop strap with a stitching at the line strap second end, wherein the stitching is located between the IV line-receiving area and the hook-loop strap interface area;
(e) wherein the hook-loop strap first end is capable of being wrapped around the desired location of the patient and the hook-loop strap bottom surface adjustably attaches to the hook-loop strap interface area; and
(f) wherein the line strap is capable of being folded over the IV line-receiving area and over the one or more IV lines, and wherein the IV line strap bottom surface is capable of adjustably attaching to the hook-loop strap top surface.

10. The IV line stabilization device of claim 9, wherein the line strap bottom surface comprises an anti-slip material and the IV line-receiving area comprises an anti-slip material.

11. The IV line stabilization device of claim 10 wherein a first grip is attached to the hook-loop strap first end and a second grip is attached to the line strap first end.

12. An IV line stabilization device used for stabilizing one or more IV lines to a desired location on a body of a patient undergoing IV therapy, comprising:
(a) an adjustable and flexible hook-loop strap comprising a hook-loop strap top surface, a hook-loop strap bottom surface, a hook-loop strap first end, a hook-loop strap second end, wherein the hook-loop strap top surface further comprises an IV line-receiving area capable of receiving one or more of the one or more IV lines, and a hook-loop strap interface area;
(b) wherein the hook-loop strap top surface comprises a hook fastener material and the hook-loop strap bottom surface comprises a loop fastener material;
(c) a flexible line strap attachable to the hook-loop strap, comprising a line strap top surface, a line strap bottom surface, a line strap first end, and a line strap second end, wherein the line strap bottom surface comprises loop fastener material;
(d) a first grip attached to the hook-loop strap first end;
(e) a second grip attached to the line strap first end;
(f) wherein the line strap bottom surface at the line strap second end is adjustably attachable to the hook-loop strap top surface;
(g) wherein the hook-loop strap first end is capable of being wrapped around the desired location of the patient and the hook-loop strap bottom surface adjustably attaches to the hook-loop strap interface area; and
(h) wherein the line strap is capable of being positioned over the IV line-receiving area and over the one or more IV lines and the line strap bottom surface is capable of being adjustably attached to the hook-loop strap top surface.

13. The IV line stabilization device of claim 12 wherein the line strap bottom surface at the line strap second end and at the line strap first end are removably attachable to the hook-loop strap top surface.

14. The IV line stabilization device of claim 12 wherein the line strap bottom surface at the line strap second end is fixably attached to the hook-loop strap top surface.

15. The IV line stabilization device of claim 14 wherein the line strap bottom surface at the line strap second end is fixably attached to the hook-loop strap top surface by sewn stitching or gluing.

16. The IV line stabilization device of claim 12, wherein the line strap bottom surface comprises an anti-slip material.

17. The IV line stabilization device of claim 12, wherein the IV line-receiving area comprises an anti-slip material.

18. The IV line stabilization device of claim 12 wherein the hook-loop strap further comprises:
(a) a flexible hook strap, comprising a hook strap top surface, a hook strap bottom surface, a hook strap first end, and a hook strap second end, wherein the hook strap top surface comprises the hook fastener material;
(b) a flexible loop strap, comprising a loop strap top surface, a loop strap bottom surface, a loop strap first end, and a loop strap second end, wherein the loop strap top surface comprises the loop fastener material; and
(c) wherein the hook strap bottom surface and the loop strap bottom surface are aligned and adjoined to form an adjustable and flexible hook-loop strap assembly.

19. The IV line stabilization device of claim 12, wherein the hook-loop strap further comprises a unitary hook-loop strap of uniform construction, further comprising a unitary hook-loop strap top surface, a unitary hook-loop strap bottom surface, a unitary hook-loop strap first end, a unitary hook-loop strap second end, wherein the unitary hook strap top surface comprises the hook fastener material and wherein the unitary hook strap bottom surface comprises the loop fastener material.

20. A method of stabilizing one or more IV lines to a desired location on a body of a patient undergoing IV therapy, wherein a catheter has been inserted into a vein of the patient at a catheter insertion site and the one or more IV lines has been connected to a valve of the catheter, comprising the steps of:
(a) placing a flexible and adjustable hook-loop strap on the body of a patient located proximately to but not over the catheter insertion site;
(i) wherein the hook-loop strap comprises a hook-loop strap top surface, a hook-loop strap bottom surface, a hook-loop strap first end, a hook-loop strap second end, wherein the hook-loop strap top surface further comprises an IV line-receiving area capable of receiving one or more of the one or more IV lines, and a hook-loop strap interface area;
(ii) wherein the hook-loop strap top surface comprises a hook fastener material and the hook-loop strap bottom surface comprises a loop fastener material;
(iii) a flexible line strap attachable to the hook-loop strap, comprising a line strap top surface, a line strap bottom surface, a line strap first end, and a line strap second end, wherein the line strap bottom surface comprises loop fastener material;
(iv) a first grip attached to the hook-loop strap first end;
(v) a second grip attached to the line strap first end;
(vi) wherein the line strap bottom surface at the line strap second end is attachable to the hook-loop strap top surface;
(vii) wherein the hook-loop strap first end is capable of being wrapped around the desired location of the patient and the hook-loop strap bottom surface adjustably attaches to the hook-loop strap interface area;
(viii) wherein the line strap is capable of being positioned over the IV line-receiving area and over the one or more IV lines and the line strap bottom surface is capable of being adjustably attached to the hook-loop strap top surface;
(b) wrapping the hook-loop strap around the desired location of the patient;
(c) adjustably attaching the hook-loop strap bottom surface at the hook-loop strap first end to the hook-loop strap interface area so that the hook-loop strap is fitted to the patient;
(d) placing the one or more IV lines in the IV line-receiving area;
(e) placing the line strap over the one or more IV lines and over the IV line-receiving area; and
(f) adjustably securing the line strap bottom surface to the hook-loop strap top surface.
